(19) United States
(12) Patent Application Publication
(10) Pub. No.: US 2025/0353746 A1
(43) Pub. Date: Nov. 20, 2025

(54) Magnetic Plasma System for Infusing Various Compounds with Nitric Oxide for Consumable, Medical and Cosmetic Products

(71) Applicant: AJPlasmatech LLC, Collegeville, PA (US)

(72) Inventors: Jennifer Blackbay, Collegeville, PA (US); Jeffrey Brooks, Oley, PA (US); Yevhen Novosolov, Philadelphia, PA (US); Marianna Diloyan, Furlong, PA (US)

(21) Appl. No.: 18/666,786

(22) Filed: May 16, 2024

(51) Int. Cl. C01B 21/32; A61K 33/00

(52) U.S. Cl. CPC C01B 21/32; A61K 33/00

(57) ABSTRACT

A system and method for infusing a compound with nitric oxide that is produced on-demand. A volume of a compound is held in a container. A magnetic plasma arc generator is provided that is connected to an air source or sources of nitrogen gas and oxygen gas. The plasma arc generator reacts the nitrogen gas with the oxygen gas to produce nitric oxide. The nitric oxide exits the output of the plasma arc generator and is directly routed into the compound within the container. The nitric oxide bubbles through the compound, therein infusing the compound with fresh nitric oxide. The compound is then rapidly sealed in packaging to seal the nitric oxide with the infused compound.

### BACKGROUND OF THE INVENTION

### 1. Field of the Invention

In general, the present invention relates to systems and methods that combine nitric oxide with topical compounds for the treatment of skin. More particularly, the present invention relates to the production of the nitric oxide and the methodology of introducing the nitric oxide into the various compounds without chemical degradation.

### 2. Prior Art Description

Nitric oxide is a molecule that is produced naturally within the body and is important for many aspects of proper health. Nitric oxide is a vasodilator, meaning it relaxes the inner muscles of the blood vessels causing them to widen and increase circulation. Accordingly, nitric oxide enables blood, nutrients, and oxygen to travel to every part of the body effectively and efficiently.

In the skin, nitric oxide is known to enhance skin quality in terms of wrinkles, lines pores, and pigment. Nitric oxide adds antimicrobial benefits to a compound. As a result, compounds that contain nitric oxide are used to treat conditions such as acme by reducing active pustules and comedones, and by improving the appearance of mild to moderate scars. The antimicrobial properties and scar repair properties of nitric oxide also make it a useful ingredient in topical wound treatments. Furthermore, n trio oxide increases moisture and the barrier functions of the skin by reducing inflammation and increasing keratinization.

Since nitric oxide has many beneficial effects for skin, nitric oxide has been added to many topical creams, serums, and cosmetics. The use of nitric oxide in a skin cream is exemplified in International Patent Publication No. WO 2012/027827 to Farber. Nitric oxide is also an active ingredient for topically applied medical products, such as eye drops. Lastly, nitric oxide is also added to medicines and health supplements that are consumed.

There are problems associated with adding nitric oxide to topical compounds, medicines, and supplements. Nitric oxide is a gas at ambient conditions, therefore nitric oxide must be purchased in canisters. If inhaled, undiluted nitric oxide is hazardous. Furthermore, pure nitric oxide can cost many hundreds of dollars per cylinder. In addition, the shipping, storage, infrastructure, and regulatory requirements for hazardous gases are complex. Further still, nitric oxide has free electrons that rapidly react to the prevalent elements of iron, oxygen, and water. In the presence of iron, nitric oxide has a half-life of less than one minute. Thus, iron and steel canisters and transfer pipes cannot be used. If exposed to air and/or water, the nitric oxide is converted into nitrates and nitrites. The reactiveness is dependent upon temperature. Hot nitric oxide combines with atmospheric oxygen to produce nitrogen dioxide, with a ninety percent conversion rate of less than one minute. Nitric oxide also has a tendency to react with itself and revert into nitrogen gas and oxygen gas over time. As such, nitric oxide degrades and becomes diluted over time. If nitric oxide of a high purity is required, the nitric oxide must be freshly produced. Fresh nitric oxide is far more expensive than general nitric oxide readily available in commerce.

Due to the need for fresh nitric oxide and the high cost of nitric oxide, many manufacturers consider making nitric oxide in-house on an as-needed basis. In commercial settings, nitric oxide is traditionally produced by the oxidation of ammonia at 750° C.-900° C., using the reaction expressed below:

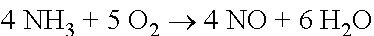

However, the reaction is not one hundred percent efficient, and some unreacted ammonia remains in the end product. The residual ammonia must be removed before the nitric oxide can be used within a topical compound because the ammonia would make the compound malodorous and could make the compound caustic. Combining the costs and complexities of producing nitric oxide on demand, few companies find producing nitric oxide more economical than buying nitric oxide in the marketplace.

A need therefore exists for a system and method where a manufacturer can produce and utilize fresh nitric oxide on demand without incurring the large costs and complexities of reducing nitric oxides from ammonia. A need also exists for a system and method to utilize the nitric oxide in production within moments of its creation and without having to store the nitric oxide or expose the nitric oxide to ambient atmosphere. These needs are met by the present invention as described and claimed below.

### SUMMARY OF THE INVENTION

The present invention is a system and method for infusing a compound with nitric oxide that is produced on-demand. A volume of a compound is held in a container. The container can be a retail packaging container or a vat that is used to fill retail packaging containers.

A magnetic plasma arc generator is provided that is connected to an air source or sources of nitrogen gas and oxygen gas. The plasma arc generator reacts the nitrogen gas with the oxygen gas to produce nitric oxide. The nitric oxide exits the output of the plasma arc generator and is directly routed into the compound within the container. In this manner, the nitric oxide has little chance to degrade prior to its use. The nitric oxide bubbles through the compound, therein infusing the compound with fresh nitric oxide. The compound is then rapidly sealed in packaging to seal the nitric oxide with the infused compound.

The nitric oxide can be cooled prior to being infused into the compound. The heat taken from the nitric oxide can be used to heat the compound being infused. This warms the compound and enables it to absorb nitric oxide more readily.

### BRIEF DESCRIPTION OF THE DRAWINGS

For a better understanding of the present invention, reference is made to the following description of exemplary embodiments thereof, considered in conjunction with the accompanying drawings, in which:

FIG. 1 is a schematic diagram illustrating the primary components for a first exemplary embodiment of the present invention system designed for a medium scale production run;

FIG. 2 is a schematic diagram illustrating the primary components for a second exemplary embodiment of the present invention system designed for a large scale production run; and

FIG. 3 is a schematic diagram illustrating the primary components for a second exemplary embodiment of the present invention system designed for a small scale production run.

### DETAILED DESCRIPTION OF THE DRAWINGS

Although the present invention system and methodology can be embodied in many ways, only three exemplary embodiments are illustrated and described. The exemplary embodiments are being shown for the purposes of explanation and description. The exemplary embodiments are selected in order to set forth some of the best modes contemplated for the invention. The illustrated embodiments, however, are merely exemplary and should not be considered limitations when interpreting the scope of the appended claims.

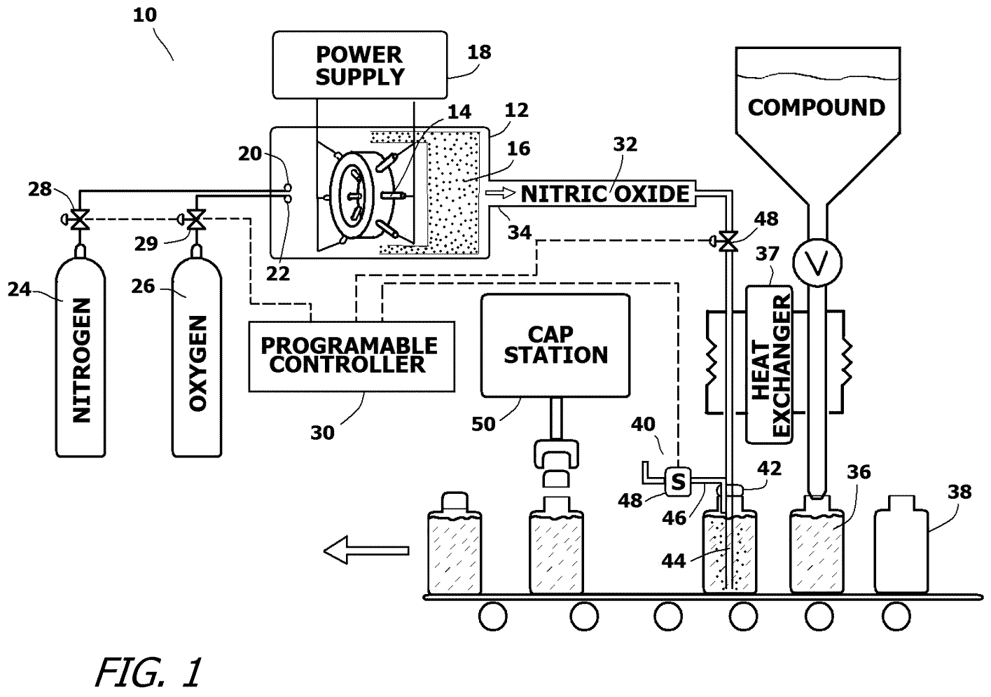

Referring to FIG. 1, an overview of the present invention production system 10 is shown. The illustrated production system 10 is for a manufacturer that has an automated factory facility. The production system 10 preferably contains a magnetic plasma arc generator 12. However, other types of plasma generators can also be used. Within the magnetic plasma arc generator 12 are one or more sets of opposed electrodes 14. A reaction catalyst 16 can be present, but is not required. The electrodes 14 are connected to a high voltage power supply 18. When the power supply 18 is activated, electric arcs are created between the electrodes 14 within the magnetic plasma arc generator 12. The arc creates plasma from any gases passing through the arc. The reaction catalyst 16, if used in the magnetic plasma arc generator 12 is preferably selected from a catalyst group that includes tungsten oxide, molybdenum oxide, chromic oxide, tantalum oxide, magnesium oxide, and copper chromite. However, other catalysts effective in the promotion of Reaction 1 below can also be used.

The magnetic plasma arc generator 12 can have a single intake for air, wherein the magnetic plasma arc generator reacts the nitrogen and oxygen in the air. However, in air, nitrogen and oxygen are not present in equal amounts. To promote a more efficient reaction, the magnetic plasma arc generator 12 in the shown embodiment has two gas intakes 20, 22. The first gas intake 20 is connected to a source of nitrogen (N₂) gas 24. The second gas intake 22 is connected to a source of oxygen (O₂) gas 26. The nitrogen gas 24 and the oxygen gas 26 are introduced into the magnetic plasma arc generator 12 in equal amounts, by molecule. The flow of nitrogen gas 24 and oxygen gas 26 are controlled by variable flow valves 28, 29. The variable flow valves 28, 29 are operated by a programmable controller 30.

The power supply 18 is activated and electric arcs are produced between the electrodes 14 in the magnetic plasma arc generator 12. As the nitrogen gas 24 and the oxygen gas 26 are introduced into the magnetic plasma arc generator 12, the arc raises the temperature of both the nitrogen gas 24 and the oxygen gas 26 to over 1000 degrees Celsius, therein producing plasmas. The nitrogen gas 24 and oxygen gas 26 react as plasmas to form nitric oxide 32 as indicated by Reaction 1 below.

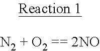

The magnetic plasma arc generator 12 has an output 34 for the nitric oxide 32 that is produced. The exiting nitric oxide 32 is hot, having an exit temperature near 1000 degrees Celsius. At this temperature, the nitric oxide is highly reactive and too hot for use. To cool the nitric oxide 32, the nitric oxide 32 passes through one or more heat exchangers 37 that cool the nitric oxide 32 to an operating temperature of that is below one hundred degrees Celsius. The heat collected from the heat exchanger 37 can be used to heat the compound 36 being infused with the nitric oxide 32.

The magnetic plasma arc generator 12 is located in a production facility that produces and packages a retail product, such as a topical cream, medication, or supplement. In the production facility, containers 38 are filled with the compounds 36 that are to be infused with the nitric oxide 32. The compounds 36 can be creams, serums, lotions, cosmetics, medications and/or nutritional supplements. In the illustrated embodiment, the containers 38 are shown as bottles. It will be understood that the containers 38 can be tubes, jars, pill casings, or any other container that is used to package topically applied products, medications and/or supplements.

In the production facility, the containers 38 are filled with the compound 36. Once the containers 38 are filled, the containers 38 pass through a gas buffer station 40. In the gas buffer station 40, each container 38 is temporarily sealed with a stopper 42 that contains an injection lead 44 and a vent lead 46. The injection lead 44 extends into the container 38 to the bottom of the compound 36. A control valve 48 introduces a select volume of the nitric oxide 32 into the container 38. The control valve 48 is operated by the programmable controller 30. The nitric oxide 32 bubbles through the compound 36, therein infusing the nitic oxide 32 into the compound 36. The nitric oxide 32 is fresh, having been produced only moments before use. From the moment of its production, the nitric oxide 32 has never been exposed to air or any other reactive material or atmosphere. As such, the nitric oxide 32 remains near pure. The only contaminants that are present in trace amounts are unreacted nitrogen gas 24 and oxygen gas 26, which are harmless to the compound 36.

As the nitric oxide 32 permeates through the compound 36, the unabsorbed gases are collected at the vent lead 46. At the vent lead 46, the pressure of the nitric oxide 32 can be monitored to ensure that the proper amount of nitric oxide 32 is being injected. Furthermore, one or more gas analyzers 49 can be provided to detect the presence of nitrogen and/or oxygen in the gas being vented. The gas analyzers 49 are connected to the programmable controller 30. If too much nitrogen and/or oxygen is being vented, then the programmable controller 30 can adjust the variable flow valves 28, 29 to alter the inputs of the nitrogen gas 24 and the oxygen gas 26. If the gas exiting the container is merely excess nitric oxide, that gas can be channeled into the output 34 of the magnetic plasma arc generator 12.

Once the compound 36 has been infused with the nitric oxide 32, the containers 38 are immediately advanced to a capping station 50 where the containers 38 are sealed. Once sealed, the nitric oxide 32 is trapped in the containers 38 and is isolated from ambient atmosphere. As a result, the nitric oxide 32 remains stable and has time to fully dissolve into the compound 36.

As soon as the last of the containers 38 is infused, the production of nitric oxide 32 can be stopped. Nitrogen gas 24 and oxygen gas 26 are atmospheric gases and are inexpensive and easy to obtain. As such, the running cost of producing the nitric oxide is the sum of the electricity costs for running the magnetic plasma arc generator 12 and the costs of the input gasses 24, 26. In many circumstances, these costs are far lower than purchasing fresh nitric oxide. Furthermore, there is no question that the nitric oxide 32 being produced is both fresh and of high purity. Furthermore, the size of the magnetic plasma arc generator 12 can be scaled to the needs of a production facility. For small facilities or small production runs, the magnetic plasma arc generator 12 can be a simple tabletop unit, as is later shown. Large production facilities can use large magnetic plasma arc generators that are built into the infrastructure, as is explained below.

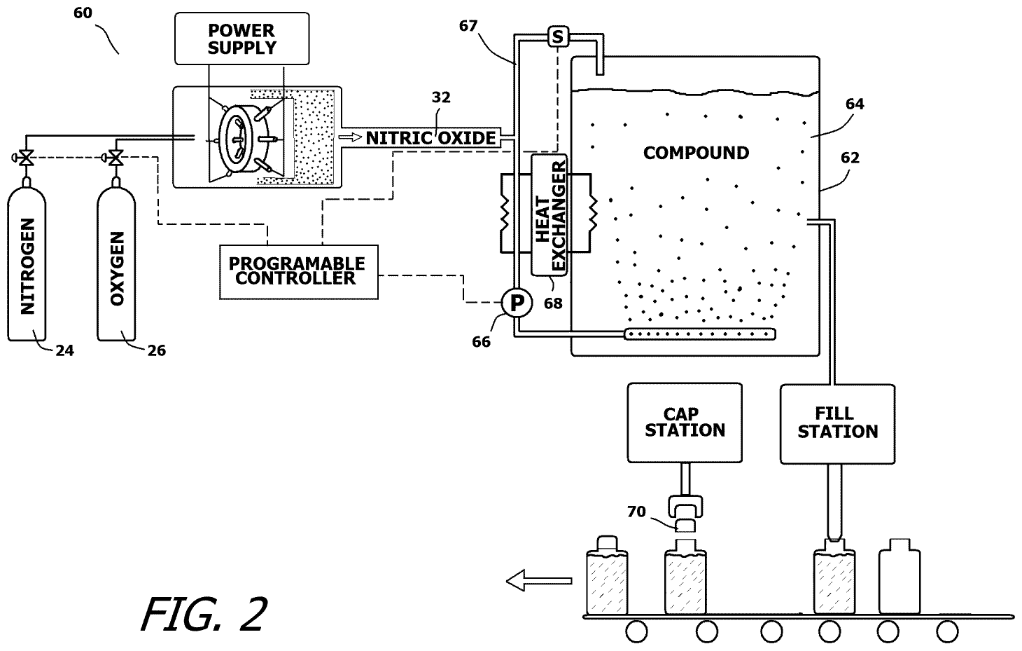

Referring to FIG. 2, an embodiment of the present invention production system 60 is shown that is optimized for large production runs. Such a system may be utilized by a large pharmaceutical company that needs to produce large volumes of product at with very high quality standards. In this embodiment, air is not used due to quality standards. Sources of nitrogen gas 24 and oxygen gas 26 are again used in place of air.

A magnetic plasma arc generator 12 converts the nitrogen gas 24 and the oxygen gas 26 into nitric oxide 32 in the same manner as has previously been described. However, in this embodiment, the nitric oxide 32 is infused into a large vat 62 that contains a full production run of a compound 64. In this embodiment, the compound 64, or at least some ingredients of the compound 64, are used to fill the vat 62. Using a pump 66, the nitric oxide 32 is pumped into the bottom of the vat 62. The nitric oxide 32 permeates through the compound 64, therein infusing the compound 64 with the nitric oxide 32. The gases that collect at the top of the vat 62 are analyzed for both composition and pressure to actively control the magnetic plasma arc generator 12 and the pump 66. If running properly, only nitric oxide 32 will collect at the top of the vat 62. As indicated by loop line 67, the nitric oxide 32 that collects at the top of the vat 62 can be reintroduced into the system 60. The nitric oxide 32 passes through a heat exchanger 68 so it can achieve a usable temperature. The heat removed from the nitric oxide 32 can be used to heat the compound 64 in the vat 62. The warmer the compound 64, the more nitric oxide 32 can be absorbed by the compound 64.

Once the compound 64 in the vat 62 is infused with the nitric oxide 32, the infused compound 64 can be bottled in containers 68. The bottling process is immediately followed with a capping process that seals the containers 68 with a cap 70. As a result, the infused compound 64 is only exposed to ambient atmosphere for a few seconds during processing. This limited exposure produces little to no detrimental effects. The nitric oxide 32 remains fresh and has time to dissolve into the compound 64 as the compound is labeled, packaged, and otherwise readied for retail sale.

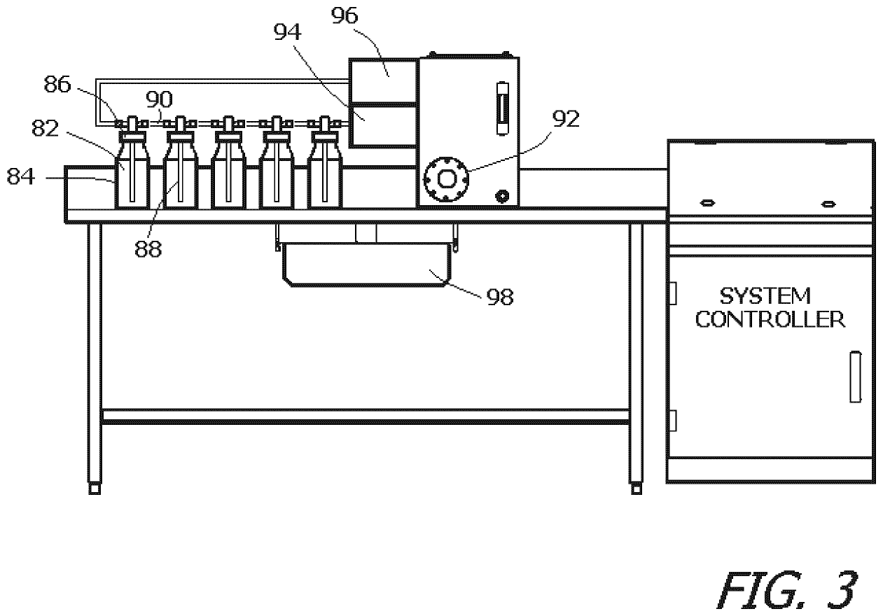

Referring to FIG. 3, an embodiment of the present invention production system 80 is shown that is configured for small runs of product. Such a system 80 can be utilized by small companies that do not have bottling facilities and produce small batches of product. In this embodiment, a compound 82 is filled into one, or a small number, of containers 84. The containers 84 can be filled by hand or can be filled in a separate machine. Once the containers 84 are filled with the compound 82, the containers 84 are temporarily sealed with injector caps 86. Each injector cap 86 has an injector tube 88 that extends into the compound 82 in the container 84. Each injector cap 86 also has a vent tube 90. The injector tubes 86 and the vent tubes 90 are daisy-chained so that the vent tube 90 of one injector cap 86 supplies the injector tube 86 of the next subsequent injector cap 86.

In previous embodiments, nitric oxide was produced by reacting nitrogen gas with oxygen gas. The nitrogen gas and the oxygen gas were separately supplied. In this embodiment, purity is not of the highest importance and air is the source of both the nitrogen and oxygen. Ambient air is approximately 78 percent nitrogen and 21 percent oxygen.

A small magnetic plasma arc generator 92 is provided. The magnetic plasma arc generator 92 is sixed for tabletop use and may contain only a single electrode and a single cathode. The plasma arc generator 92 draws air from the atmosphere and reacts the air to produce nitric oxide.

The nitric oxide passes through a first gas analyzer 94 to ensure that sufficient amounts of nitric oxide are being produced and that no detrimental compounds are present. The nitric oxide flows through the various containers 84 and infuses into the compounds 82 held within those containers 84. In a closed loop the remaining nitric oxide is returned to the magnetic plasma arc generator 92.

As the remnant nitric oxide returns to the magnetic plasma arc generator 92, the nitric oxide passes through a second gas analyzer 96. The second gas analyzer 96 can determine if the remnant nitric oxide is too much or too little. In this manner, the flow of air provided to the magnetic plasma arc generator 92 can be dynamically adjusted.

If the compound 82 in the containers 84 need agitation and/or heating, a hot plate and magnetic stirring device 98 can be provided that acts upon the container 84 during the infusion process. Once the infusion process is complete, the containers 84 are manually removed and replaced. If the containers 84 are the final packaging containers, then the containers 84 are quickly capped. If the compound 82 is poured into secondary packaging containers, this process is performed in a rapid manner to minimize exposure of the compound to ambient conditions.

It will be understood that the embodiments of the present invention that are illustrated and described are merely exemplary and that a person skilled in the art can make many variations to those embodiments. All such embodiments are intended to be included within the scope of the present invention as defined by the claims.

What is claimed is:

1. A method for infusing a compound with nitric oxide, comprising:
providing a volume of said compound;
providing a plasma arc generator, wherein said plasma arc generator has an output that leads into said volume of said compound;
reacting nitrogen gas and oxygen gas in said plasma arc generator to generate nitric oxide;
directing said nitric oxide directly from said plasma arc generator into said compound, wherein said nitric oxide infuses into said compound.

2. The method according to claim 1, further including cooling said nitric oxide as said nitric oxide travels from said plasma arc generator to said compound.

3. The method according to claim 2, wherein cooling said nitric oxide includes passing said nitric oxide through a heat exchanger that removes heat from said nitric oxide.

4. The method according to claim 3, wherein said heat removed from said nitric oxide is used to heat said compound.

5. The method according to claim 1, wherein reacting said nitrogen gas and oxygen gas in said plasma arc generator includes introducing said nitrogen gas and said oxygen gas into said plasma arc generator through at least one variable flow valve.

6. The method according to claim 5, wherein gases escape from said compound while said nitric oxide is introduced into said compound, wherein said method further includes monitoring said gases escaping said compound.

7. The method according to claim 6, further including adjusting said at least one variable flow valve to reduce said nitrogen gas entering said plasma arc generator should free nitrogen, in excess of a threshold value, be detected in said gases escaping said compound.

8. The method according to claim 6, further including adjusting said at least one variable flow valve to reduce said oxygen gas entering said plasma arc generator should free oxygen, in excess of a threshold value, be detected in said gases escaping said compound.

9. The method according to claim 1, further including filling said compound into package containers prior to directing said nitric oxide into said compound.

10. The method according to claim 9, further including sealing said package containers, therein trapping said nitric oxide within said package containers with said compound.

11. The method according to claim 1, further including filling said compound into package containers after directing said nitric oxide into said compound.

12. A system for infusing a compound with nitric oxide, comprising:
a container holding a volume of said compound;
a plasma arc generator having an output that leads into said container;
a source of nitrogen gas that leads into said plasma arc generator;
a source of oxygen gas that leads into said plasma arc generator;
wherein said nitrogen gas and said oxygen gas react in said plasma arc generator to produce nitric oxide, and wherein said nitric oxide is directed through said output directly into said container, therein causing said nitric oxide to infuse into said compound within said container.

13. The system according to claim 12, further including a heat exchanger for cooling said nitric oxide as said nitric oxide travels from said plasma arc generator to said container.

14. The system according to claim 13, wherein said heat exchanger transfers heat from said output of said plasma arc generator to said compound within said container.

15. The system according to claim 12, further including variable flow valves for selectively controlling said nitrogen gas and said oxygen gas entering said plasma arc generator.

16. The system according to claim 15, further including at least one gas analyzer for monitoring gases that escape from said compound in said container when said nitric oxide is introduced into said container.

17. The system according to claim 16, further including a programmable controller that selectively adjusts said variable flow valves to reduce said nitrogen gas entering said plasma arc generator should free nitrogen, in excess of a threshold value, be detected in said gases that escape from said compound.

18. The method according to claim 16, further including a programmable controller that selectively adjusts said variable flow valves to reduce said oxygen gas entering said plasma arc generator should free oxygen, in excess of a threshold value, be detected in said gases that escape from said compound.

19. A system for infusing a compound with nitric oxide, comprising:
a container holding a volume of said compound;
a plasma arc generator having an output that leads into said container, wherein said plasma arc generator has an air intake;
wherein said air intake introduces air into said plasma arc generator that contains nitrogen gas and oxygen gas, and wherein said nitrogen gas and said oxygen gas react in said plasma arc generator to produce nitric oxide, and
wherein said nitric oxide is directed through said output directly into said container, therein causing said nitric oxide to infuse into said compound within said container.

20. The system according to claim 19, further including at least one gas analyzer for monitoring gases that escape from said compound in said container when said nitric oxide is introduced into said container.
